(19) United States
(12) Patent Application Publication
(10) Pub. No.: US 2026/0091032 A1
(43) Pub. Date: Apr. 2, 2026

(54) AMORPHOUS SOLID DISPERSIONS

(71) Applicant: INTRA-CELLULAR THERAPIES, INC., Bedminster, NJ (US)

(72) Inventor: Peng Li, New Milford, NJ (US)

(73) Assignee: INTRA-CELLULAR THERAPIES, INC., Bedminster, NJ (US)

(21) Appl. No.: 19/292,836

(22) Filed: Aug. 6, 2025

Related U.S. Application Data
(63) Continuation of application No. 18/468,199, filed on Sep. 15, 2023, now Pat. No. 12,472,178.
Application No. 18/468,199 is a division of application No. 17/655,143, filed on Mar. 16, 2022, now Pat. No. 11,872,223.
Application No. 17/655,143 is a continuation of application No. 16/221,162, filed on Dec. 14, 2018, now Pat. No. 11,311,536.
Application No. 16/221,162 is a continuation-in-part of application No. PCT/US2017/054962, filed on Oct. 3, 2017.
(60) Provisional application No. 62/407,285, filed on Oct. 12, 2016.

(51) Int. Cl. A61K 31/4985; A61K 9/00; A61K 9/10; A61K 9/16; A61K 9/20; A61K 9/48; A61K 47/38; A61P 3/04; A61P 25/06; A61P 25/18; A61P 25/22; A61P 25/24; A61P 25/28; C07D 471/16

(52) U.S. Cl. CPC A61K 31/4985; A61K 9/0019; A61K 9/0024; A61K 9/0053; A61K 9/1617; A61K 9/1635; A61K 9/1641; A61K 9/1652; A61K 9/2013; A61K 9/2027; A61K 9/2031; A61K 9/2054; A61K 9/4858; A61K 9/4866; A61K 47/38; A61P 3/04; A61P 25/06; A61P 25/18; A61P 25/22; A61P 25/24; A61P 25/28; A61K 9/10; C07D 471/16

(57) ABSTRACT

The disclosure provides new, stable, pharmaceutically acceptable amorphous solid dispersions of 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one, together with methods of making and using them, and pharmaceutical compositions comprising them.

### CROSS-REFERENCE TO RELATED APPLICATIONS

This application is a continuation-in-part, filed under 35 U.S.C. § 111, of international application PCT/US2017/054962, filed on Oct. 3, 2017, which claims priority to and the benefit of U.S. Provisional Application Ser. No. 62/407,285, filed on Oct. 12, 2016, the content of each of which are hereby incorporated by reference in their entireties.

### FIELD

This disclosure relates to certain novel amorphous solid dispersion formulations of a substituted heterocycle fused gamma-carboline, the manufacture of such dispersions, pharmaceutical compositions comprising such dispersions, and uses thereof, e.g., in the treatment of diseases or abnormal conditions involving or mediated by the 5-HT_{2A} receptor, serotonin transporter (SERT), and/or dopamine D₁/D₂ receptor signaling pathways.

### BACKGROUND

1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (sometimes referred to as 4-((6bR,10aS)-3-methyl-2,3,6b,9,10,10a-hexahydro-1H-pyrido[3′,4′:4,5]pyrrolo[1,2,3-de]quinoxalin-8(7H)-yl)-1-(4-fluorophenyl)-1-butanone, or Lumateperone or as ITI-007), has the following structure:

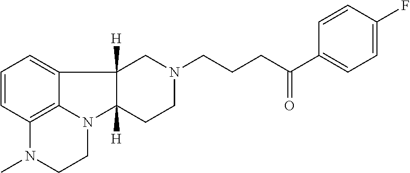 [SMILES: CN1CCN2c3c(cccc31)[C@@H]1CN(CCCC(=O)c3ccc(F)cc3)CC[C@@H]12]

ITI-007 is a potent 5-HT_{2A} receptor ligand (Ki=0.5 nM) with strong affinity for dopamine (DA) D2 receptors (Kᵢ=32 nM) and the serotonin transporter (SERT) (Kᵢ=62 nM), but negligible binding to receptors associated with cognitive and metabolic side effects of antipsychotic drugs (e.g., H1 histaminergic, 5-HT_{2C}, and muscarinic receptors). ITI-007 is currently in clinical trials, i.e., for the treatment of schizophrenia. While ITI-007 is a promising drug, its production and formulation present distinct challenges. In free base form, ITI-007 is an oily, sticky solid, with poor solubility in water. Making salts of the compound has proven to be unusually difficult. A hydrochloride salt form of ITI-007 was disclosed in U.S. Pat. No. 7,183,282, but this salt was hygroscopic and shows poor stability. A toluenesulfonic acid addition salt (tosylate) of ITI-007 was finally identified and described in WO 2009/114181. Both of these publications are incorporated by reference in their entirety.

Nevertheless, there remains a need for alternative stable, pharmaceutically acceptable solid forms of ITI-007 which can be readily incorporated into galenic formulations.

It has been disclosed that for a number of drugs, amorphous forms exhibit different dissolution characteristics, and in some cases different bioavailability patterns, compared to crystalline forms of the same drug. For some therapeutic indications, one bioavailability pattern may be favored over another. For example, an amorphous form of Cefuroxime axetil exhibits higher bioavailability than the crystalline form. Thus, amorphous solid dispersions are a promising alternative to traditional crystalline active pharmaceutical ingredients.

Pure amorphous drug forms tend to be unstable. As amorphous forms are thermodynamically unstable relative to the corresponding crystal forms, it is well known that amorphous forms would revert back to the stable crystalline form. This usually occurs during storage under various humidity and temperature conditions. Therefore, in order to utilize the amorphous form of a drug, it is necessary to stabilize it to inhibit crystallization of the drug active during the period of product storage.

Discovering suitable excipients that will stabilize the amorphous form of a pharmaceutical drug is a challenge, as some excipients will chemically react with the drug or promote its decomposition, while other excipients will form uniform solid dispersions that are not physically stable, not chemically stable or both.

### SUMMARY

Given the difficulties involved in making salts of ITI-007, it was decided to explore whether the compound could be formulated as a physically and chemically stable amorphous solid dispersion. An extensive screen of excipients was undertaken, using various combinations of agents at different ratios and using different production methods. Dispersions were evaluated based on physical appearance and texture, X-ray powder diffraction (XRPD), modulated differential scanning calorimetry (mDSC), thermogravimetric analysis (TGA), and high-performance liquid chromatography (HPLC). Sixteen potential excipients were screened under a total of forty-four conditions, and three pharmaceutically acceptable amorphous solid dispersions were discovered.

The present disclosure therefore provides amorphous solid dispersions of ITI-007 in free base form or in tosylate salt form, in admixture with one or more excipients, such as stabilizing excipients.

In one aspect, the present disclosure provides three particularly stable amorphous solid dispersions of ITI-007 free base comprising (1) ITI-007 free base at a 5:95 to 50:50 weight ratio to cellulose acetate excipient; (2) ITI-007 free base at a 25:75 to 75:25 weight ratio to cellulose acetate phthalate excipient; and (3) ITI-007 free base at a 25:75 to 75:25 weight ratio to hydroxypropyl methyl cellulose phthalate excipient. The present disclosure further provides several amorphous solid dispersions of ITI-007 tosylate, optionally further comprising an antioxidant and/or a surfactant.

The disclosure thus provides novel amorphous solid dispersion forms of ITI-007 free base and tosylate salt, which dispersions are especially advantageous for use in the preparation of galenic formulations, together with methods of making and using the same.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 depicts an overlay of X-ray powder diffraction patterns for dispersions of ITI-007 free base with cellulose acetate.

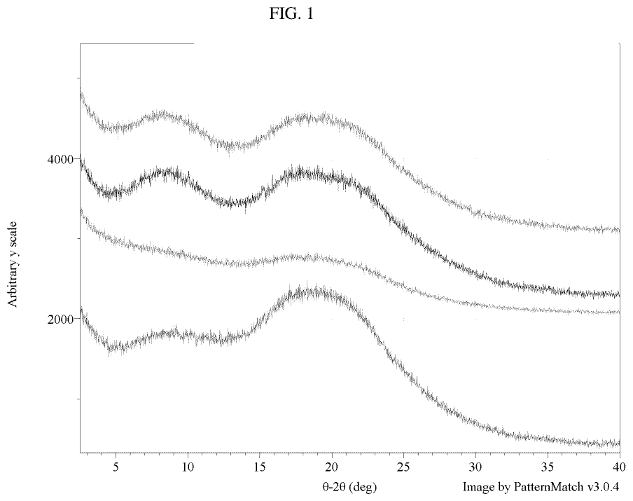

FIG. 2 depicts an overlay of X-ray powder diffraction patterns for dispersions of ITI-007 free base with cellulose acetate phthalate.

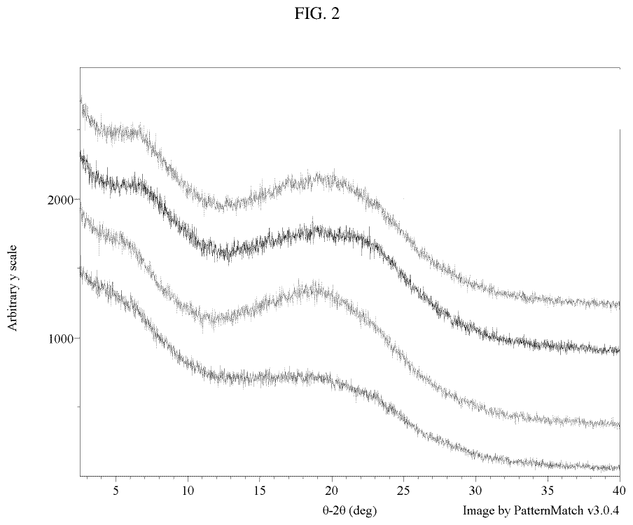

FIG. 3 depicts an overlay of X-ray powder diffraction patterns for dispersions of ITI-007 free base with hydroxypropyl methyl cellulose phthalate (grade 55) (HPMC-P).

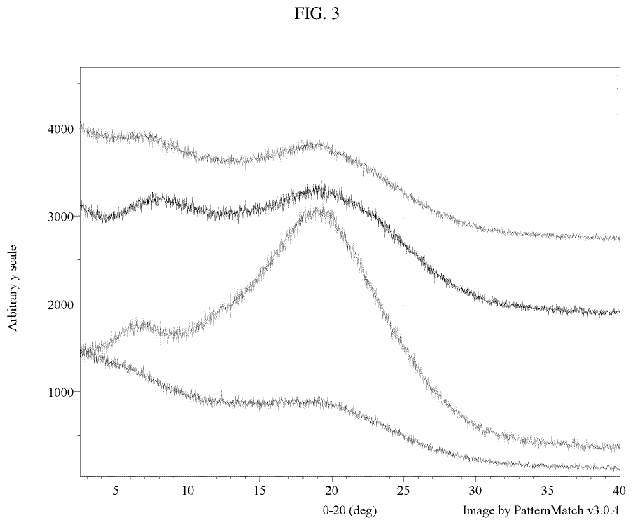

For each of FIGS. 1, 2 and 3, the top pattern is the 25:75 ITI-007 free base/excipient dispersion as-generated; the second pattern is the 25:75 dispersion post-stress; the third pattern is the 50:50 ITI-007 free base/excipient dispersion as-generated; the bottom pattern is the 50:50 dispersion post-stress.

FIG. 4 depicts mDSC and TGA thermograms for a 25:75 dispersion of ITI-007 free base with cellulose acetate.

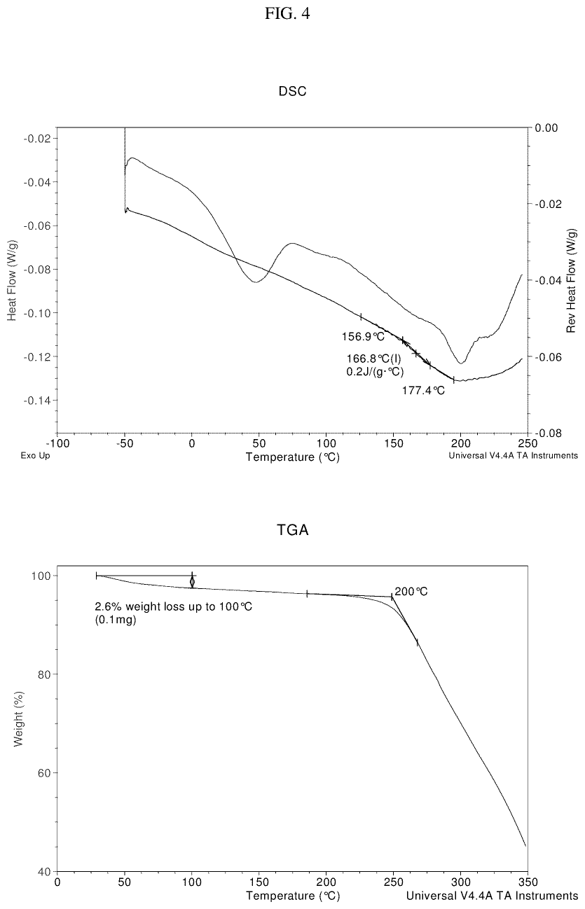

FIG. 5 depicts mDSC and TGA thermograms for a 50:50 dispersion of ITI-007 free base with cellulose acetate phthalate.

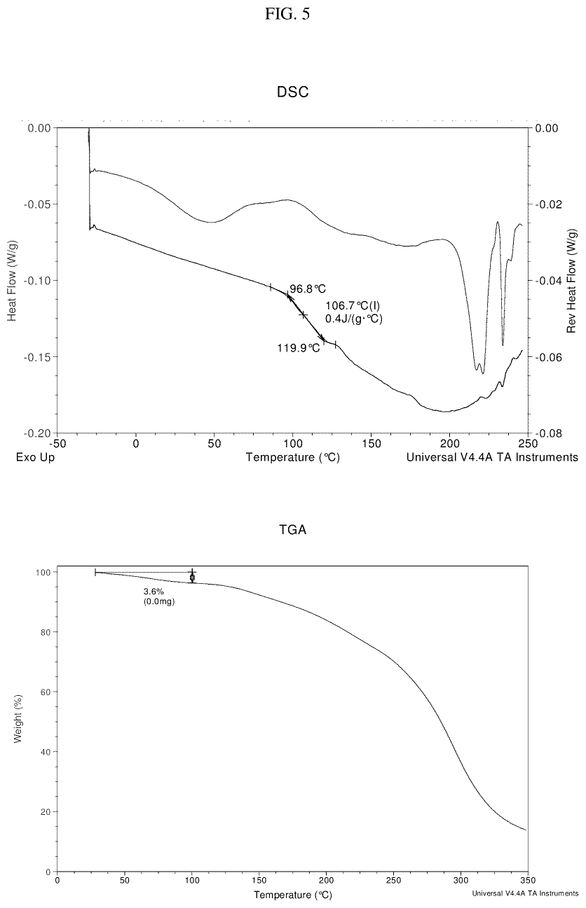

FIG. 6 depicts mDSC and TGA thermograms for a 50:50 dispersion of ITI-007 free base with HPMC-P.

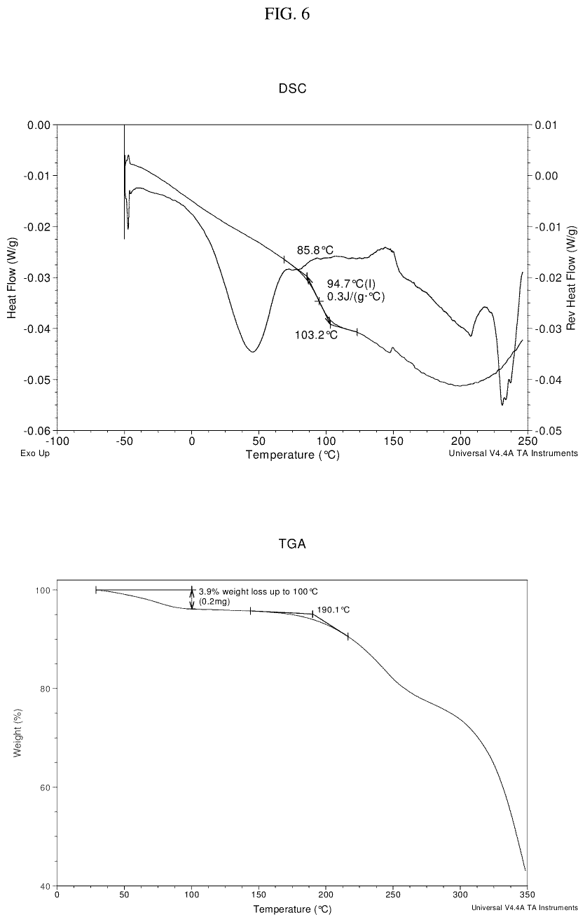

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present disclosure provides amorphous solid dispersions of 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007), either in free base form or in tosylate salt form, in admixture with one or more excipients, such as stabilizing excipients

In a first embodiment, the present disclosure provides 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) free base in the form of an amorphous solid dispersion comprising cellulose acetate excipient in a ratio of 5:95 to 50:50 ITI-007 free base to cellulose acetate (Dispersion 1). The present disclosure further provides the following Compositions:
1.1. Dispersion 1, wherein the dispersion comprises ITI-007 free base and cellulose acetate in a weight ratio of 5:95 up to 50:50, but excluding the ratio 50:50.
1.2. Dispersion 1 or 1.1, wherein the dispersion comprises ITI-007 free base and cellulose acetate in a weight ratio of 5:95 to 49:51, e.g., 5:95 to 45:55, or 10:90 to 40:60, or 15:85 to 35:65, or 20:80 to 30:70, or 22:78 to 28:82, or 23:77 to 27:83, or 24:76 to 26:74, or about 25:75.
1.3. Any foregoing dispersion, wherein the dispersion is x-ray amorphous, e.g., as shown by XRPD analysis.
1.4. Any foregoing dispersion, wherein the X-ray diffraction pattern is free of peaks characteristic of the excipient.
1.5. Any foregoing dispersion, wherein the dispersion shows a single glass transition temperature (T_{g}) above 75° C., e.g., at a temperature above 100° C., or at a temperature above 150° C., e.g., as shown by mDSC analysis.
1.6. Dispersion 1.5, wherein the dispersion shows a single glass transition temperature above 160° C., or between 165° C. and 170° C., or at about 167° C.
1.7. Any foregoing dispersion, wherein the dispersion shows a change in heat capacity (ΔCp) of 0.1 to 0.5 J/g-° C., e.g., from 0.2 to 0.3 J/g-° C., or about 0.2 J/g-° C., e.g., as shown by mDSC.
1.8. Any foregoing dispersion, wherein the dispersion shows less than 10% weight loss up to a temperature of 100° C., e.g., as shown by TGA analysis.
1.9. Dispersion 1.8, wherein the dispersion shows less than 8% weight loss up to a temperature of 100° C., e.g., less than 7% weight loss, or less than 6% weight loss, or less than 5% weight loss, or less than 4% weight loss, or less than 3% weight loss, up to a temperature of 100° C.
1.10. Any foregoing dispersion, wherein the dispersion shows no changes in appearance or texture after 7 days at 75% relative humidity at 40° C.
1.11. Any foregoing dispersion, wherein the dispersion shows greater than 90% chemical stability of ITI-007 after 7 days at 75% relative humidity at 40° C., e.g., as judged by HPLC.
1.12. Dispersion 1.11, wherein the dispersion shows greater than 95%, or greater than 96%, or greater than 97% or greater than 98%, or greater than 99% chemical stability of ITI-007 after 7 days at 75% relative humidity at 40° C.
1.13. Any foregoing dispersion, wherein the dispersion is manufactured by a method comprising dissolving ITI-007 free base and the selected excipient in a suitable solvent or mixture of solvents and removing the solvent, e.g., by lyophilizing the solution, to obtain the amorphous solid dispersion.
1.14. Dispersion 1.13, wherein the solvent or mixture of solvents is selected from dioxane, methanol, ethanol, tetrahydrofuran, acetone, and mixtures thereof.
1.15. Dispersion 1.13, wherein the solvent or mixture of solvents is selected from dioxane, methanol or a dioxane/methanol mixture, e.g., a 90:10 to 98:2 ratio of dioxane to methanol, or a 92:8 to 95:5 ratio, or about a 93:7 ratio of dioxane to methanol.
1.16. Any foregoing dispersion, wherein the dispersion exhibits any combination of characteristics as described in 1.1-1.15.

In a second embodiment, the present disclosure provides ITI-007 free base in the form of an amorphous solid dispersion comprising cellulose acetate phthalate excipient in a ratio of 25:75 to 75:25 ITI-007 free base to cellulose acetate phthalate (Dispersion 2). The present disclosure further provides the following Compositions:
2.1. Dispersion 2, wherein the dispersion comprises ITI-007 free base and cellulose acetate phthalate in a weight ratio of from 25:75 up to 75:25, but excluding the ratios 25:75 and 75:25.
2.2. Dispersion 2 or 2.1, wherein the dispersion comprises ITI-007 free base and cellulose acetate phthalate in a weight ratio of 26:74 to 74:26, e.g., 30:70 to 70:30, or 35:65 to 65:35, or 40:60 to 60:40, or 42:58 to 58:42, or 44:56 to 56:44, or 45:55 to 55:45, or 47:53 to 53:47, or 48:52 to 52:48, or 49:51 to 51:49, or about 50:50.
2.3. Any foregoing dispersion, wherein the dispersion is x-ray amorphous, e.g., as shown by XRPD analysis.
2.4. Any foregoing dispersion, wherein the X-ray diffraction pattern is free of peaks characteristic of the excipient.
2.5. Any foregoing dispersion, wherein the dispersion shows a single glass transition temperature (T_{g}) above 75° C., e.g., at a temperature above 85° C., or at a temperature above 95° C., e.g., as shown by mDSC analysis.
2.6. Dispersion 2.5, wherein the dispersion shows a single glass transition temperature above 100° C., or between 105° C. and 115° C., or at about 107° C.
2.7. Any foregoing dispersion, wherein the dispersion shows a change in heat capacity (ΔCp) of 0.1 to 0.6 J/g-° C., e.g., from 0.2 to 0.5 J/g-° C., or about 0.4 J/g-° C., e.g., as shown by mDSC.
2.8. Any foregoing dispersion, wherein the dispersion shows less than 10% weight loss up to a temperature of 100° C., e.g., as shown by TGA analysis.
2.9. Dispersion 2.8, wherein the dispersion shows less than 8% weight loss up to a temperature of 100° C., e.g., less than 7% weight loss, or less than 6% weight loss, or less than 5% weight loss, or less than 4% weight loss, or less than 3% weight loss, up to a temperature of 100° C.
2.10. Any foregoing dispersion, wherein the dispersion shows no changes in appearance or texture after 7 days at 75% relative humidity at 40° C.
2.11. Any foregoing dispersion, wherein the dispersion shows greater than 90% chemical stability of ITI-007 after 7 days at 75% relative humidity at 40° C., e.g., as judged by HPLC.
2.12. Dispersion 2.11, wherein the dispersion shows greater than 95%, or greater than 96%, or greater than 97% or greater than 98%, or greater than 99% chemical stability of ITI-007 after 7 days at 75% relative humidity at 40° C.
2.13. Any foregoing dispersion, wherein the dispersion is manufactured by a method comprising dissolving ITI-007 free base and the selected excipient in a suitable solvent or mixture of solvents and removing the solvent, e.g., by lyophilizing the solution, to obtain the amorphous solid dispersion.
2.14. Dispersion 2.13, wherein the solvent or mixture of solvents is selected from dioxane, methanol, ethanol, tetrahydrofuran, acetone, and mixtures thereof.
2.15. Dispersion 2.13, wherein the solvent or mixture of solvents is selected from dioxane, methanol or a dioxane/methanol mixture, e.g., a 90:10 to 98:2 ratio of dioxane to methanol, or a 92:8 to 95:5 ratio, or about a 93:7 ratio of dioxane to methanol.
2.16. Any foregoing dispersion, wherein the dispersion exhibits any combination of characteristics as described in 2.1-2.15.

In a third embodiment, the present disclosure provides ITI-007 free base in the form of an amorphous solid dispersion comprising hydroxypropyl methyl cellulose phthalate (HPMC-P) excipient in a ratio of 25:75 to 75:25 ITI-007 free base to HPMC-P (Dispersion 3). The present disclosure further provides the following Compositions:
3.1. Dispersion 3, wherein the dispersion comprises ITI-007 free base and HPMC-P in a weight ratio of from 25:75 up to 75:25, but excluding the ratios 25:75 and 75:25.
3.2. Dispersion 3 or 3.1, wherein the dispersion comprises ITI-007 free base and HPMC-P in a weight ratio of 26:74 to 74:26, e.g., 30:70 to 70:30, or 35:65 to 65:35, or 40:60 to 60:40, or 42:58 to 58:42, or 44:56 to 56:44, or 45:55 to 55:45, or 47:53 to 53:47, or 48:52 to 52:48, or 49:51 to 51:49, or about 50:50.
3.3. Any foregoing dispersion, wherein the dispersion is x-ray amorphous, e.g., as shown by XRPD analysis.
3.4. Any foregoing dispersion, wherein the X-ray diffraction pattern is free of peaks characteristic of the excipient.
3.5. Any foregoing dispersion, wherein the dispersion shows a single glass transition temperature (T_{g}) above 75° C., e.g., at a temperature above 80° C., or at a temperature above 85° C., e.g., as shown by mDSC analysis.
3.6. Dispersion 3.5, wherein the dispersion shows a single glass transition temperature above 90° C., or between 92° C. and 98° C., or at about 95° C. 3.7. Any foregoing dispersion, wherein the dispersion shows a change in heat capacity (ΔCp) of 0.1 to 0.5 J/g-° C., e.g., from 0.2 to 0.4 J/g-° C., or about 0.3 J/g-° C., e.g., as shown by mDSC.
3.8. Any foregoing dispersion, wherein the dispersion shows less than 10% weight loss up to a temperature of 100° C., e.g., as shown by TGA analysis.
3.9. Dispersion 3.8, wherein the dispersion shows less than 8% weight loss up to a temperature of 100° C., e.g., less than 7% weight loss, or less than 6% weight loss, or less than 5% weight loss, or less than 4% weight loss, or less than 3% weight loss, up to a temperature of 100° C.
3.10. Any foregoing dispersion, wherein the dispersion shows no changes in appearance or texture after 7 days at 75% relative humidity at 40° C.
3.11. Any foregoing dispersion, wherein the dispersion shows greater than 90% chemical stability of ITI-007 after 7 days at 75% relative humidity at 40° C., e.g., as judged by HPLC.
3.12. Dispersion 3.11, wherein the dispersion shows greater than 95%, or greater than 96%, or greater than 97% or greater than 98%, or greater than 99% chemical stability of ITI-007 after 7 days at 75% relative humidity at 40° C.
3.13. Any foregoing dispersion, wherein the dispersion is manufactured by a method comprising dissolving ITI-007 free base and the selected excipient in a suitable solvent or mixture of solvents and removing the solvent, e.g., by lyophilizing the solution, to obtain the amorphous solid dispersion.
3.14. Dispersion 3.13, wherein the solvent or mixture of solvents is selected from dioxane, methanol, ethanol, tetrahydrofuran, acetone, and mixtures thereof.
3.15. Dispersion 3.13, wherein the solvent or mixture of solvents is selected from dioxane, methanol or a dioxane/methanol mixture, e.g., a 90:10 to 98:2 ratio of dioxane to methanol, or a 92:8 to 95:5 ratio, or about a 93:7 ratio of dioxane to methanol.
3.16. Any foregoing dispersion, wherein the dispersion exhibits any combination of characteristics as described in 3.1-3.15.

In a fourth embodiment, the present disclosure provides ITI-007 tosylate salt in the form of an amorphous solid dispersion comprising a stabilizing excipient and optionally further comprising an anti-oxidant and/or a surfactant (Dispersion 4). For example, the stabilizing excipient is an excipient which stabilizes the amorphous from of ITI-007 tosylate to prevent conversion of the amorphous form to the crystal form. The present disclosure further provides the following Compositions:
4.1. Dispersion 4, wherein the dispersion comprises ITI-007 tosylate salt and a stabilizing excipient selected from the group consisting of cellulose acetate, cellulose acetate phthalate, methacrylate/methyl acrylate copolymer, hydroxypropyl cellulose, hydroxypropyl methyl cellulose, hydroxypropyl methyl cellulose acetate succinate (HPMC-AS), hydroxypropyl methyl cellulose phthalate (HPMC-P), polyvinyl acetate, polyvinyl pyrrolidone, polyvinyl pyrrolidone/vinyl acetate copolymer, and polyethylene glycol/polyvinyl acetate/polyvinylcaprolactam copolymer.
4.2. Dispersion 4 or 4.1, wherein the composition comprises ITI-007 tosylate salt in admixture with a single stabilizing excipient.
4.3. Dispersion 4 or 4.1, wherein the composition comprises ITI-007 tosylate salt in admixture with two stabilizing excipients.
4.4. Dispersion 4 or any of 4.1 to 4.3, wherein the dispersion comprises ITI-007 tosylate salt in admixture with one or more stabilizing excipients in a weight ratio of 25:75 to 75:25, e.g., 26:74 to 74:26, or 30:70 to 70:30, or 35:65 to 65:35, or 40:60 to 60:40, or 42:58 to 58:42, or 44:56 to 56:44, or 45:55 to 55:45, or 47:53 to 53:47, or 48:52 to 52:48, or 49:51 to 51:49, or about 50:50.
4.5. Dispersion 4 or any of 4.1 to 4.3, wherein the dispersion comprises ITI-007 tosylate salt in admixture with one or more stabilizing excipients in a weight ratio of 5:95 to 50:50, e.g., 5:95 to 49:51, or 5:95 to 45:55, or 10:90 to 40:60, or 15:85 to 35:65, or 20:80 to 30:70, or 22:78 to 28:82, or 23:77 to 27:83, or 24:76 to 26:74, or about 25:75.
4.6. Dispersion 4 or any of 4.1 to 4.3, wherein the dispersion comprises ITI-007 tosylate salt in admixture with one or more stabilizing excipients in a weight ratio of 50:50 to 95:5, e.g., 51:49 to 95:5, or 55:45 to 95:5, or 60:40 to 90:10, or 65:45 to 85:15, or 70:30 to 80:20 or about 75:25.
4.7. Any foregoing dispersion, wherein the stabilizing excipient comprises one or more of cellulose acetate, cellulose acetate phthalate, hydroxypropyl cellulose, hydroxypropyl methyl cellulose, hydroxypropyl methyl cellulose acetate succinate (HPMC-AS), and hydroxypropyl methyl cellulose phthalate (HPMC-P).
4.8. Any foregoing dispersion, wherein the stabilizing excipient comprises one or more of cellulose acetate, cellulose acetate phthalate, and hydroxypropyl methyl cellulose phthalate.
4.9. Any foregoing dispersion, further comprising an anti-oxidant.
4.10. Dispersion 4.9, wherein the anti-oxidant is selected from one or more of tocopherol, butylated hydroxytoluene (BHT), propyl gallate (OPG), and ascorbic acid.
4.11. Dispersion 4.9 or 4.10, wherein the dispersion comprises the antioxidant in an amount of 0.1 to 10% by weight, e.g., 0.5 to 5% or 0.5 to 3% by weight of the dispersion.
4.12. Any foregoing dispersion, further comprising a surfactant, for example, an anionic or cationic or neutral surfactants, such as a surfactant which stabilizes the amorphous form ITI-007 tosylate (e.g., to prevent conversion to an ITI-007 tosylate crystal form).
4.13. Any foregoing dispersion, wherein the dispersion is x-ray amorphous, e.g., as shown by XRPD analysis.
4.14. Any foregoing dispersion, wherein the X-ray diffraction pattern is free of peaks characteristic of the excipient(s).
4.15. Any foregoing dispersion, wherein the dispersion shows a single glass transition temperature (T_{g}) above 75° C., e.g., at a temperature above 80° C., or at a temperature above 90° C., or a temperature about 100° C., or at a temperature above 110° C., e.g., as shown by mDSC analysis.
4.16. Any foregoing dispersion, wherein the dispersion shows a change in heat capacity (ΔCp) of 0.1 to 2.0 J/g-° C., e.g., from 0.1 to 1.5 J/g-° C., or 0.1 to 1.0 J/g-° C., or 0.1 to 0.5 J/g-° C., e.g., as shown by mDSC.
4.17. Any foregoing dispersion, wherein the dispersion shows less than 20% weight loss up to a temperature of 150° C., e.g., as shown by TGA analysis.
4.18. Dispersion 4.13, wherein the dispersion shows less than 15% weight loss up to a temperature of 150° C., e.g., less than 15% weight loss up to a temperature of 100° C., or less than 10% weight loss up to a temperature of 150° C., or less than 10% weight loss up to a temperature of 100° C., or less than 5% weight loss up to a temperature of 150° C., or less than 5% weight loss up to a temperature of 100° C.
4.19. Any foregoing dispersion, wherein the dispersion shows no changes in appearance or texture after 7 days at 75% relative humidity at 40° C.
4.20. Any foregoing dispersion, wherein the dispersion shows greater than 85% chemical stability of ITI-007 after 7 days at 75% relative humidity at 40° C., e.g., as judged by HPLC.
4.21. Dispersion 4.20, wherein the dispersion shows greater than 90%, or greater than 95%, or greater than 96%, or greater than 97% or greater than 98%, or greater than 99% chemical stability of ITI-007 after 7 days at 75% relative humidity at 40° C.
4.22. Any foregoing dispersion, wherein the dispersion is manufactured by a method comprising dissolving ITI-007 tosylate salt and the selected excipient(s) in a suitable solvent or mixture of solvents and removing the solvent, e.g., by lyophilizing the solution or evaporating the solvent (e.g., by rotary evaporation), to obtain the amorphous solid dispersion.
4.23. Dispersion 4.22, wherein the solvent or mixture of solvents is selected from dioxane, methanol, ethanol, tetrahydrofuran, acetone, and mixtures thereof.
4.24. Dispersion 4.23, wherein the solvent or mixture of solvents is selected from dioxane, methanol or a dioxane/methanol mixture, e.g., a 90:10 to 98:2 ratio of dioxane to methanol, or a 92:8 to 95:5 ratio, or about a 93:7 ratio of dioxane to methanol.
4.25. Dispersion 4.22, 4.23, or 4.24, wherein the method further comprises adding an anti-oxidant or a surfactant to the solvent mixture prior to removing the solvent.
4.26. Any foregoing dispersion, wherein the dispersion exhibits any combination of characteristics as described in 4.1-4.25.

Dispersion 4, and any of 4.1-4.26, may comprise an antioxidant to improve the stability of the ITI-007 tosylate salt amorphous form. In some embodiments, the anti-oxidant is selected from one or more of tocopherol, butylated hydroxytoluene (BHT), propyl gallate (OPG), and ascorbic acid.

In other embodiments the antioxidant may be selected from additional suitable antioxidants, such as butylated hydroxyanisole (BHA), tert-Butylhydroquinone (TBHQ), carotenoids, glutathione, sodium metabisulfite, sodium ethylenediaminetetraacetate, cysteine, methionine, sesamol, and citric acid.

Dispersion 4, and any of 4.1-4.26, may comprise a surfactant to improve the stability of the ITI-007 tosylate salt amorphous form. In some embodiments, the surfactant is a surfactant which stabilizes the amorphous form ITI-007 tosylate (e.g., to prevent conversion to an ITI-007 tosylate crystal form). In some embodiments, the surfactant is selected from one or more of an anionic or cationic or neutral surfactant.

In some embodiments of the present disclosure, Dispersion 4, or any of 4.1-4.26, may further comprise any other excipient which can prevent or inhibit the formation of the crystal form of ITI-007 tosylate, or which can prevent or inhibit the conversion of the amorphous form of ITI-007 tosylate to a crystal form of ITI-007 tosylate. Such excipients may include polymers, gums, surfactants, wetting agents, drying agents, pH modifiers, fillers, disintegrants, coatings, binders, or any other suitable pharmaceutically acceptable excipients.

In a second aspect, the present disclosure provides a process (Process 1) for the production of Dispersion 1, et seq., or Dispersion 2, et seq., or Dispersion 3, et seq., comprising the steps of:
(a) combining 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) free base with the selected excipient in a suitable solvent or mixture of solvents, e.g., selected from dioxane, methanol, ethanol, tetrahydrofuran, acetone, and mixtures thereof; and
(b) removing the solvent and recovering the amorphous solid dispersion thus formed, e.g., by lyophilization of the solution.

In another embodiment of the second aspect, the present disclosure provides a process (Process 2) for the production of Dispersion 4, et seq., comprising the steps of:
(a) combining 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) tosylate salt, e.g., monotosylate, optionally in crystal form, with the selected excipient or excipients in a suitable solvent or mixture of solvents, e.g., selected from dioxane, methanol, ethanol, tetrahydrofuran, acetone, and mixtures thereof; and
(b) removing the solvent and recovering the amorphous solid dispersion thus formed, e.g., by lyophilization of the solution or evaporating the solvent (e.g., by rotary evaporation).
   In some embodiments, Process 2 further comprises the step of adding one or more anti-oxidants and/or one or more surfactants to the solvent or mixture of solvents in step (a). In some embodiments, the anti-oxidant is selected from one or more of tocopherol, butylated hydroxytoluene (BHT), propyl gallate (OPG), and ascorbic acid. In some embodiments, the one or more surfactants may comprise an anionic or cationic or neutral surfactant. For example, the surfactant may be a surfactant which stabilizes the amorphous form ITI-007 tosylate, such as to prevent conversion of the amorphous form to an ITI-007 tosylate crystal form).

In other embodiments, the antioxidant may be selected from additional suitable antioxidants, such as butylated hydroxyanisole (BHA), tert-Butylhydroquinone (TBHQ), carotenoids, glutathione, sodium metabisulfite, sodium ethylenediaminetetraacetate, cysteine, methionine, sesamol, and citric acid.

In another embodiment of the second aspect, the solvent or mixture of solvents for Process 1 is selected from dioxane, methanol or a dioxane/methanol mixture, e.g., a 90:10 to 98:2 ratio of dioxane to methanol, or a 92:8 to 95:5 ratio, or about a 93:7 ratio of dioxane to methanol, optionally wherein the solvent is removed by lyophilization.

Solid dispersion, as used herein, refers to the dispersion of an active pharmaceutical ingredient, i.e., ITI-007, in an inert excipient or matrix (carrier), where the active ingredient could exist in a finely crystalline, solubilized or amorphous state. The excipient in a solid dispersion is typically a polymer. The most important role of the polymer in a solid dispersion is to reduce the molecular mobility of the pharmaceutical active to avoid phase separation and re-crystallization of the active during storage. The amorphous form of the active is associated with a higher energy state as compared to its crystalline counterpart, and therefore, significantly less external energy is required to effect dissolution (e.g., in the gastrointestinal tract or elsewhere in the body).

In a third aspect, the present disclosure provides a pharmaceutical composition (Composition 1) comprising Dispersion 1, et seq., or Dispersion 2, et seq., or Dispersion 3, et seq., or Dispersion 4, et seq., in combination or association with a pharmaceutically acceptable diluent or carrier. In some embodiments, the pharmaceutical composition is in the form of a tablet or capsule for oral administration. In some embodiments, the pharmaceutical composition is in the form of a depot formulation for use as a long-acting injectable (LAI). The pharmaceutical composition may further comprise any suitable pharmaceutically acceptable excipient, such as: diluents such as starches, pregelatinized starches, lactose, powdered celluloses, microcrystalline celluloses, dicalcium phosphate, tricalcium phosphate, mannitol, sorbitol, xylitol, sugar and the like; binders such as acacia, guar gum, tragacanth, gelatin, polyvinylpyrrolidones such as polyvinylpyrrolidones(PVP K-30,K-90), poly (vinyl pyrrolidone-co-vinyl acetate) (PVP-VA) and the like, hydroxypropyl celluloses, hydroxypropyl methylcellulose, cellulose acetate, hydroxypropyl methylcellulose acetate succinate (HPMC-AS) and the like; disintegrants such as starches, sodium starch glycolate, pregelatinized starches, crospovidones, croscarmellose sodium and the like; lubricants such as stearic acid, magnesium stearate, zinc stearate and the like; glidants such as colloidal silicon dioxide and the like; solubility or wetting enhancers such as anionic or cationic or neutral surfactants; maltodextrin, complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like; and film formers, plasticizers, colorants, flavoring agents, sweeteners, viscosity enhancers, preservatives, antioxidants, and the like.

In another embodiment of the third aspect, the composition may further comprise one or more anti-oxidants, for example, tocopherol, butylated hydroxytoluene (BHT), propyl gallate (OPG), and ascorbic acid, or the like. The inclusion of an anti-oxidant may further improve the chemical stability of the dispersions by preventing oxidative chemical degradation of the ITI-007 active. In another embodiment, the dispersion itself is formulated to include such an anti-oxidant.

In another embodiment of the third aspect, the composition may further comprise any excipient which can prevent or inhibit the formation of the crystal form of ITI-007 free base or ITI-007 tosylate, or which can prevent or inhibit the conversion of the amorphous form of ITI-007 free base or ITI-007 tosylate to a crystal form thereof. Such excipients may include polymers, gums, surfactants, wetting agents, drying agents, pH modifiers, fillers, disintegrants, coatings, binders, or any other suitable pharmaceutically acceptable excipients. Such excipients include any one or more of the excipients described in paragraph [0033] above.

In another aspect, the present disclosure provides Dispersion 1, et seq., or Dispersion 2, et seq., or Dispersion 3, et seq., or Dispersion 4, et seq., or a pharmaceutical composition comprising Dispersion 1, et seq., or Dispersion 2, et seq., or Dispersion 3, et seq., or Dispersion 4, et seq., e.g., Composition 1, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression, anxiety, psychosis, schizophrenia, migraine, obsessive-compulsive disorder, sexual disorders, depression, schizophrenia, migraine, attention deficit disorder, attention deficit hyperactivity disorder, obsessive-compulsive disorder, sleep disorders, conditions associated with cephalic pain, social phobias, or dementia.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression, anxiety, psychosis, schizophrenia, migraine, obsessive-compulsive disorder, sexual disorders, depression, schizophrenia, migraine, attention deficit disorder, attention deficit hyperactivity disorder, obsessive-compulsive disorder, sleep disorders, conditions associated with cephalic pain, social phobias, or dementia, comprising administering to a patient in need thereof a therapeutically effective amount of Dispersion 1, et seq., or Dispersion 2, et seq., or Dispersion 3, et seq., or a pharmaceutical composition comprising Dispersion 1, et seq., or Dispersion 2, et seq., or Dispersion 3, et seq, or Dispersion 4, et seq., e.g., Composition 1.

### EXAMPLES

The following equipment and methods are used to isolate and characterize the exemplified co-crystal forms:

X-ray powder diffraction (XRPD): The X-ray powder diffraction studies are performed using a PANalytical X′Pert PRO MPD diffractometer using an incident beam of Cu radiation produced using an Optix long, fine-focus source. An elliptically graded multilayer mirror is used to focus Cu Kα X-ray radiation through the specimen and onto the detector. Prior to analysis, a silicon specimen is analyzed to verify the observed position of the Si (111) peak (consistent with the NIST-certified position, NIST SM 640e). A specimen of the sample is sandwiched between 3-micron thick films and analyzed in transmission geometry. A beam-stop, short antiscatter extension, and antiscatter knife edge is used to minimize the background generated by the air. Soller slits for the incident and diffracted beams are used to minimize broadening from axial divergence. Diffraction patterns are collected using a scanning position-sensitive detector (X'Celerator) located 240 mm from the specimen. Data Collector software v. 2.2b is used for analysis.

Thermogravimetry (TGA) analysis: TGA is performed using a TA Instruments Q5000 or Discovery thermogravimetric analyzer. The sample is placed in an aluminum sample pan and is inserted into the TG furnace. Samples are heated from ambient temperature to 250° C. at a rate of 10° C./minute. Nickel and Alumel are used as the calibration standards.

Modulated Differential Scanning Calorimetry (mDSC): mDSC data is obtained on a TA Instruments Q2000 or 2920 differential scanning calorimeter equipped with a refrigerated cooling system. Temperature calibration is performed using NIST traceable indium metal. The sample is placed into an aluminum T-zero DSC pan, covered with a lid, and the weight is accurately recorded. A weighed aluminum pan configured as the sample pan is placed on the reference side of the cell. Typically, the start temperature is −50° C. and the end temperature is 250° C., with a modulation amplitude of ±1° C. and a 50 second period with an underlying heating rate of 2° C. per minute.

High performance liquid chromatography (HPLC): The high-performance liquid chromatography analyses are performed using an Agilent 1100 series liquid chromatograph equipped with a diode array detector, degasser, quaternary pump, and an auto sampler. The column is a 4.6×100 mm CSH C18 column with 2.5-micron packing (XSelect) running with a 0.1% TFA in water mobile phase A and a 0.1% TFA in acetonitrile mobile phase B, at a flow rate of 0.500 mL/minute. The gradient runs from 95% A to 73% over the first 22 minutes, followed by 6 minutes at 73% A, and followed by 73% A to 30% A over the next 22 minutes. The column temperature is set to 15.0° C., and the detector wavelength is 254 nm with a bandwidth of 100 nm and a reference wavelength of 360 nm. The injection volume is 2.0 microliters.

### Example 1: Preparation of Free Base Dispersions

Solubility of ITI-007 free base and various excipients is first evaluated in various solvents. It is found that ITI-007 free base shows good solubility (>50 mg/mL) in acetone, ethanol, methanol, dioxane, and 2,2,2-trifluoroethanol (TFE), but relatively poor solubility (5-50 mg/mL) in tert-butanol/water mixtures. However, it is found that solutions of ITI-007 free base in TFE rapidly discolor due to decomposition of the active.

The excipients evaluated are Eudragit L100, polyvinyl acetate, polyvinylpyrrolidone-vinyl acetate copolymer, polyvinylpyrrolidone K-90, polyvinylpyrrolidone S-630, cellulose acetate, cellulose acetate phthalate, Gelucire 50/13, glyceryl monostearate, hydroxypropyl cellulose, hydroxypropyl methyl cellulose phthalate (HPMC-P), hydroxypropyl methyl acetate succinate (HPMC-AS), polyethylene glycol (PEG), PEG-100 succinate, Pluronic F-127, and Soluplus. Excipients were evaluated at one or more of the ratios 25:75, 50:50 and 75:25 ITI-007 free base to excipient.

Based on the solubility analyses, solutions of various excipients with ITI-007 free base are prepared in 3:1 acetone-ethanol. Rotary evaporation is attempted to remove the solvent, but this results in oily materials, instead of solids, in all cases.

Solid dispersions are successfully prepared by lyophilization from solutions of ITI-007 free base and excipient in either dioxane or dioxane-methanol (90:10, 91:9, 92:8, 93:7 or 94:6). Solutions are initially frozen in a dry ice/acetone bath, and then placed in a freeze dryer with the shelf pre-cooled to −75° C. Samples are dried overnight at −50° C., followed by −20° C., then 0° C. over a period of two days. Samples are then secondary dried at 20° C. for four hours, purged with nitrogen then stored in a freezer over desiccant until testing.

### Example 2: Preliminary Screen of Free Base Dispersions

Solid dispersions obtained from Example 1 are first evaluated by XRPD to determine if they are amorphous. All lyophilization samples using amorphous excipients are found to be x-ray amorphous by XRPD. Lyophilization samples using crystalline excipients (Gelucire 50/13, PEG, PEG-1000 succinate, Pluronic F-127) are found to be disordered with peaks present corresponding only to the excipient. Further observations of the appearance of the solids are shown in Table 1 below. The 50:50 ITI-007/PEG-1000 succinate dispersion is found to be very sticky and is not further evaluated.

### Example 3: Stability Evaluation of Free Base Dispersions

Solid dispersions from Example 1 are placed into uncapped clear glass vials and the vials are placed into a container maintained for seven days at 75% relative humidity and a temperature of 40° C. As a control, a sample of ITI-007 free base is analyzed in parallel. Samples were observed visually as well as by polarized light microscopy (0.8-10× magnification with crossed polarizers and a first order red compensator). Observations are shown in Table 1. The majority of samples display changes in appearance or texture, indicating the formation of physically unstable amorphous dispersions. For example, some show visible crystallization while others become sticky solids or oils.

Dispersions which are physically stable free-flowing solids are further analyzed by XRPD to confirm that they remain x-ray amorphous or disordered with excipient peaks only. The XRPD results confirm that the visually stable samples remain X-ray amorphous dispersions.

mDSC and TGA analysis is conducted on the physically stable free-flowing samples. A single glass transition temperature in mDSC supports the conclusion that the solid is a non-crystalline miscible dispersion. The two PEG dispersions show an unacceptable low-temperature glass transition at 9 or 10° C., while the glyceryl monostearate dispersion shows no glass transition. The 50:50 cellulose acetate dispersion shows two glass transition temperatures, which suggests a phase-separated material, which is unacceptable. Only the 25:75 cellulose acetate, 25:75 cellulose acetate phthalate, 50:50 cellulose acetate phthalate, 25:75 HPMC-AS, 50:50 HPMC-AS, 25:75 HPMC-P and 50:50 HPMC-P dispersions show acceptable single glass transition temperatures above 75° C.

All samples submitted to mDSC and TGA are then submitted to HPLC analysis to determine the chemical stability of the ITI-007 active agent during the seven-day study. As a control, the ITI-007 free base sample is also analyzed by HPLC. All results are normalized to the ITI-007 content shown by HPLC prior to the seven-day study. A loss of less than 5% ITI-007 by HPLC is considered satisfactory.

Both HPMC-AS dispersions, as well as the 25:75 HPMC-P dispersion show very high material losses by HPLC. The 25:75 cellulose acetate phthalate dispersion shows a low but unacceptable loss of material. Only seven dispersions produce satisfactory results: 25:75 cellulose acetate, 50:50 cellulose acetate, 50:50 cellulose acetate phthalate, 50:50 HPMC-P, 25:75 PEG, 50:50 PEG and 25:75 glyceryl stearate. These dispersions are thus chemically stable.

The combined tests results are shown in Table 1 below.

| | ITI-007/ | X-ray | Stable for 7 | | % ITI-007 |
|---|---|---|---|---|---|
| | Excipient | Amorphous | days at 40° C./ | | change by |
| Excipient | Ratio | Solids? | 75% RH | T_{g} >75° C. | HPLC |
| Cellulose | 25:75 | Yes | Yes | Yes (167° C.) | 0% |
| Acetate | 50:50 | Yes | Yes | No | −4.43% |
| | 75:25 | Yes | No | | |
| Cellulose | 25:75 | Yes | Yes | Yes (142° C.) | −5.65% |
| Acetate | 50:50 | Yes | Yes | Yes (107° C.) | −2.42% |
| Phthalate | 75:25 | Yes | No | | |
| Gelucire | 25:75 | No | No | | |
| 50/13 | 50:50 | No | No | | |
| Glyceryl | 25:75 | No | No | No | 0% |
| Monostearate | 50:50 | No | No | | |
| HPC | 25:75 | Yes | No | | |
| | 50:50 | Yes | No | | |
| HPMC-AS | 25:75 | Yes | | Yes (88° C.) | −55.29% |
| MG | 50:50 | Yes | | Yes (77° C.) | −8.76% |
| | 75:25 | Yes | No | | |
| HPMC-P | 25:75 | Yes | | Yes (123° C.) | −18.89% |
| | 50:50 | Yes | | Yes (95° C.) | −0.01% |
| | 75:25 | Yes | No | | |
| PEG | 25:75 | No | No | No | −0.30% |
| | 50:50 | No | No | No | 0% |
| | 75:25 | No | No | | |
| PEG-1000 | 25:75 | No | No | | |
| Succinate | 50:50 | No | | | |
| Pluronic | 25:75 | No | No | | |
| F-127 | 50:50 | No | No | | |
| PVAc | 25:75 | Yes | No | | |
| | 50:50 | Yes | No | | |
| PVP S-630 | 25:75 | Yes | No | | |
| | 50:50 | Yes | No | | |
| | 75:25 | Yes | No | | |
| PVP K-90 | 25:75 | Yes | No | | |
| | 50:50 | Yes | No | | |
| | 75:25 | Yes | No | | |
| PVP co-VA | 25:75 | Yes | No | | |
| | 50:50 | Yes | No | | |
| | 75:25 | Yes | No | | |
| Soluplus | 25:75 | Yes | No | | |
| | 50:50 | Yes | No | | |
| | 75:25 | Yes | No | | |
| ITI-007 only | 100:0 | No | | No | −5.97% |

Of the tested dispersions, it is found that only three are both chemically stable and physically stable: 25:75 cellulose acetate, 50:50 cellulose acetate phthalate, and 50:50 HPMC-P.

It is noted that similar experiments conducted using ITI-007 tosylate salt, instead of ITI-007 free base, result in no completely stable amorphous dispersions. These results are shown in more detail in Example 4 below. While most of the ITI-007 tosylate dispersions pass the initial screen (X-ray amorphous or showing only X-ray peaks due to the excipient), all of the resulting initial dispersions display strong physical instability (color and appearance changes, including crystallization of the active agent out of the dispersion) or chemical instability (10-68% decomposition by HPLC). For example, the 25:75 dispersion of ITI-007 tosylate with cellulose acetate produced crystallization of ITI-007 during the aging study; the 50:50 dispersion of ITI-007 tosylate with cellulose acetate phthalate showed about a 52% decrease in ITI-007 content by HPLC; and the 50:50 dispersion of ITI-007 tosylate with HPMC-P showed about a 68% decrease in ITI-007 content by HPLC. These results are unexpected because ITI-007 tosylate is chemically more stable than ITI-007 free base. Thus, it is particularly unexpected that three specific amorphous solid dispersions of ITI-007 free base are physically and chemically stable whereas the corresponding dispersions of ITI-007 tosylate are not.

### Example 4: Preparation, Preliminary Screen and Stability Evaluation of Tosylate Dispersions

The procedures described in Example 1, 2 and 3 are repeated using ITI-007 tosylate salt as the active ingredient. It is found that ITI-007 tosylate has good solubility (>50 mg/mL) in 2,2,2-trifluoroethanol (TFE) and in a 60:40 v/v mixture of tert-butanol/water, but relatively poor solubility (20-40 mg/mL) in acetone, ethanol, dioxane and other tert-butanol/water mixtures. However, it is also found that solutions of ITI-007 tosylate salt in TFE rapidly discolor due to decomposition of the active.

The same excipients are evaluated as described in Example 1. As is seen for the free base, rotary evaporation from solvent (3:1 acetone-methanol) does not result in amorphous material, but lyophilization from dioxane and dioxane/methanol mixtures results in amorphous material using each excipient tested. Screening and stability evaluations are shown in Table 2 below.

| | ITI-007/ | X-ray | Stable for 7 | | % ITI-007 |
|---|---|---|---|---|---|
| | Excipient | Amorphous | days at 40° C./ | | change by |
| Excipient | Ratio | Solids? | 75% RH | T_{g} >75° C. | HPLC |
| Cellulose | 25:75 | Yes | Some Crystal | | |
| Acetate | 50:50 | Yes | Some Crystal | | |
| Cellulose | 25:75 | Yes | Color change | Yes (131° C.) | −23.30% |
| Acetate | 50:50 | Yes | Color change | Yes (112° C.) | −52.14% |
| Phthalate | 75:25 | Yes | Texture change | | |
| Gelucire | 25:75 | No | Some Crystal | | |
| 50/13 | 50:50 | No | Some Crystal | | |
| Glyceryl | 25:75 | No | Some Crystal | | |
| Monostearate | 50:50 | No | Some Crystal | | |
| HPC | 25:75 | Yes | Texture change | | |
| | 50:50 | Yes | Texture change | | |
| | 75:25 | Yes | Texture change | | |
| HPMC-AS | 25:75 | Yes | Color change | Yes (91° C.) | −12.10% |
| MG | 50:50 | Yes | Texture change | | |
| | 75:25 | Yes | Texture change | | |
| HPMC-P | 25:75 | Yes | Color change | Yes (116° C.) | −23.11% |
| | 50:50 | Yes | Color change | Yes (94° C.) | −67.77% |
| | 75:25 | Yes | Texture change | | |
| PEG | 25:75 | No | Some Crystal | | |
| | 50:50 | No | Some Crystal | | |
| PEG-1000 | 25:75 | No | Texture change | | |
| Succinate | 50:50 | No | Texture change | | |
| Pluronic | 25:75 | No | Texture change | | |
| F-127 | 50:50 | No | Some Crystal | | |
| PVAc | 25:75 | Yes | Texture change | | |
| | 50:50 | Yes | Texture change | | |
| PVP S-630 | 25:75 | Yes | Texture change | | |
| | 50:50 | Yes | Texture change | | |
| | 75:25 | Yes | Texture change | | |
| PVP K-90 | 25:75 | Yes | Texture change | | |
| | 50:50 | Yes | Texture change | | |
| | 75:25 | Yes | Texture change | | |
| PVP co-VA | 25:75 | Yes | Texture change | | |
| | 50:50 | Yes | Texture change | | |
| Soluplus | 25:75 | Yes | Texture change | | |
| | 50:50 | Yes | Texture change | | |
| | 75:25 | Yes | Texture change | | |
| ITI-007 only | 100:0 | Yes | Some crystal | No | 0.00% |

Of the tested dispersions using ITI-007 tosylate salt, most of are found to be amorphous and under the harsh storage conditions of 75% relative humidity and a temperature of 40° C., several dispersions are observed visually to have undergone some crystallization. In addition, other dispersions are found to have undergone some minor color changes or texture changes. Further evaluation suggests, however, that these changes in physical appearance may be prevented by incorporating an anti-oxidant into the composition to prevent air-promoted oxidation, forming the dispersion into a coated tablet, encapsulating the dispersion into a gel caplet or capsule, adding excipient(s), such as polymers, to prevent partial conversion of amorphous formulation to crystallized forms, and/or admixing the dispersion with surfactant to inhibit particle agglomeration.

1. An amorphous solid dispersion comprising 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one.

2. The dispersion of claim 1, wherein the dispersion is 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) tosylate salt in the form of an amorphous solid dispersion comprising a stabilizing excipient and optionally further comprising an anti-oxidant and/or a surfactant.

3. The dispersion of claim 2, wherein the dispersion comprises ITI-007 tosylate salt and one or more stabilizing excipients selected from the group consisting of cellulose acetate, cellulose acetate phthalate, methacrylate/methyl acrylate copolymer, hydroxypropyl cellulose, hydroxypropyl methyl cellulose, hydroxypropyl methyl cellulose acetate succinate (HPMC-AS), hydroxypropyl methyl cellulose phthalate (HPMC-P), polyvinyl acetate, polyvinyl pyrrolidone, polyvinyl pyrrolidone/vinyl acetate copolymer, and polyethylene glycol/polyvinyl acetate/polyvinylcaprolactam copolymer.

4. The dispersion of claim 2, further comprising an anti-oxidant, optionally selected from one or more of tocopherol, butylated hydroxytoluene (BHT), propyl gallate (OPG), and ascorbic acid.

5. The dispersion of claim 2, further comprising a surfactant, optionally an anionic or cationic or neutral surfactant.

6. The dispersion of claim 2, wherein the dispersion is x-ray amorphous.

7. A pharmaceutical composition comprising the dispersion of claim 2, in combination or association with a pharmaceutically acceptable diluent or carrier.

8. The composition of claim 7, wherein the composition is in the form of a tablet or capsule for oral administration.

9. A method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulemia, depression, anxiety, psychosis, schizophrenia, migraine, obsessive-compulsive disorder, sexual disorders, depression, schizophrenia, migraine, attention deficit disorder, attention deficit hyperactivity disorder, obsessive-compulsive disorder, sleep disorders, conditions associated with cephalic pain, social phobias, or dementia, comprising administering to a patient in need thereof a therapeutically effective amount of a dispersion according to claim 2.

10. A process for the production of the dispersion of claim 2 comprising the steps of:
(a) combining 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) tosylate salt, e.g., monotosylate, optionally in crystal form, with the selected excipient or excipients in a suitable solvent or mixture of solvents, e.g., selected from dioxane, methanol, ethanol, tetrahydrofuran, acetone, and mixtures thereof; and
(b) removing the solvent and recovering the amorphous solid dispersion thus formed, e.g., by lyophilization of the solution or evaporating the solvent (e.g., by rotary evaporation).

11. The dispersion of claim 1, wherein the dispersion is 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) free base and:
(a) cellulose acetate excipient in a ratio of 5:95 to 50:50 ITI-007 free base to cellulose acetate; or
(b) cellulose acetate phthalate excipient in a ratio of 25:75 to 75:25 ITI-007 free base to cellulose acetate phthalate; or
(c) HPMC-P excipient in a ratio of 25:75 to 75:25 ITI-007 free base to HPMC-P.

12. (canceled)

13. (canceled)

14. (canceled)

15. (canceled)

16. (canceled)

17. (canceled)

18. (canceled)

19. (canceled)

20. The dispersion of claim 11, wherein the dispersion is manufactured by a method comprising dissolving ITI-007 free base and the selected excipient in a suitable solvent or mixture of solvents and removing the solvent to obtain the amorphous solid dispersion.

21. The dispersion of claim 20, wherein the solvent or mixture of solvents is selected from dioxane, methanol, ethanol, tetrahydrofuran, acetone, and mixtures thereof.

22. The dispersion of claim 20, wherein the solvent or mixture of solvents is selected from dioxane, methanol or a dioxane/methanol mixture.

23. The dispersion of claim 20, wherein the solvent or mixture of solvents is dioxane and methanol in a 90:10 to 98:2 ratio of dioxane to methanol, or a 92:8 to 95:5 ratio, or a 93:7 ratio of dioxane to methanol.

24. A process for the production of the dispersion of claim 11, comprising the steps of:
(a) combining 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (ITI-007) free base with the selected excipient in a suitable solvent or mixture of solvents; and
(b) removing the solvent and recovering the amorphous solid dispersion thus formed.

25. A pharmaceutical composition comprising the dispersion of claim 11, in combination or association with a pharmaceutically acceptable diluent or carrier.

26. The composition of claim 25, wherein the composition is in the form of a tablet or capsule for oral administration.

27. The composition of claim 25, wherein the composition is in the form of a depot formulation for use as a long-acting injectable (LAI).

28. A method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulemia, depression, anxiety, psychosis, schizophrenia, migraine, obsessive-compulsive disorder, sexual disorders, depression, schizophrenia, migraine, attention deficit disorder, attention deficit hyperactivity disorder, obsessive-compulsive disorder, sleep disorders, conditions associated with cephalic pain, social phobias, or dementia, comprising administering to a patient in need thereof a therapeutically effective amount of a dispersion according to claim 11.
